(19) United States
(12) Patent Application Publication
(10) Pub. No.: US 2008/0003669 A1
(43) Pub. Date: Jan. 3, 2008

(54) HYBRID AERATION MEMBRANE BIOREACTOR

(76) Inventors: Zhiming LIAO, Shenzhen (CN); Rong LI, Shenzhen (CN); Dejin JU, Shenzhen (CN); Jijun WU, Shenzhen (CN); Jingkui GUO, Shenzhen (CN); Yuhe HUANG, Shenzhen (CN); Dongsheng CAI, Shenzhen (CN); Aiguo WAN, Shenzhen (CN); Kun YU, Shenzhen (CN); Zhizhong LIU, Shenzhen (CN)

Correspondence Address: MATTHIAS SCHOLL, 14781 MEMORIAL DRIVE, SUITE 1319, HOUSTON, TX, 77079, US

(21) Appl. No.: 11/769,831

(22) Filed: Jun. 28, 2007

(30) Foreign Application Priority Data
Jun. 28, 2006 (CN) 200610061358.0

(51) Int. Cl. C12M 1/04

(52) U.S. Cl. 435/296.1

(57) ABSTRACT

The present invention teaches a hybrid aeration membrane bioreactor, comprising a bio-reaction tank; a plurality of membrane modules disposed within the bio-reaction tank; a macropore aeration apparatus comprising a first aeration pipe; a plurality of macropore heads having each a plurality of macropores, the macropore heads being disposed on the first aeration pipe; and a first gas pump; and a micropore aeration apparatus comprising a second aeration pipe; a plurality of micropore heads having each a plurality of micropores, the micropore heads being disposed on the second aeration pipe; and a second gas pump; wherein the first aeration pipe is connected to the first gas pump; the second aeration pipe is connected to the second gas pump; the macropore head is disposed below the membrane module; the micropore head is disposed near the bottom of the bio-reaction tank; and the micropores are smaller in diameter than the macropores. In accordance with the present invention, two aeration types, the macropore aeration and the micropore aeration, are combined, at the same aeration rate, the oxygen utilization rate of the membrane bioreactor is increased and thus the energy consumption for treating waste water is decreased. Therefore, the reactor is suitable for treating highly loaded organic waste water.

### CROSS-REFERENCE TO RELATED APPLICATIONS

Pursuant to 35 U.S.C. § 119 and the Paris Convention Treaty, this application claims the benefit of Chinese Patent Application No. 200610061358.0 filed Jun. 28, 2006, the contents of which are incorporated herein by reference.

### BACKGROUND OF THE INVENTION

1. Field of the Invention

The invention relates to waste water treatment equipments, and more particularly, to a membrane bioreactor for treating highly-loaded organic waste water.

2. Description of the Related Art

The membrane bioreactor (MBR) process is a waste water treatment process to integrate the modern membrane filtering technology with the conventional activated sludge process. The membrane bioreactor mainly comprises a bioreactor and a plurality of membrane separators (membrane module), wherein the bioreactor serves as a major site for degrading the pollutant substances in waste water. The membrane separator, which is generally an ultra-filtration or a micro-filtration membrane, serves mainly for separating solid and liquid and filtering certain macromolecular compounds.

The main principle in the operation of a membrane bioreactor is to replace the secondary sedimentation tank used by the conventional activated sludge process with one or more ultra-filtration or micro-filtration membranes for the solid-liquid separation. Owing to the filtration of the membrane, the sludge retention time (SRT) and the hydropower retention time (HRT) may be separately controlled so that the treatment process provides the advantages of good solid-liquid separation effect, high biochemical efficiency, stable production of high quality water, high sludge concentration, and strong impact resistance capability. Furthermore, since the secondary sedimentation tank is removed and the backflow of sludge is prevented, the equipment is compact, floor space is saved, and the investment and operational costs are decreased.

Pollution of membrane modules may occur due to microbially produced extra-celluarpolymers (EPS) or by direct adhesion of microorganisms to the membrane at their high concentrations. The pollution of membranes can be classified into internal pollution and external pollution. Internal pollution refers to the obstruction by and adsorption of substances with a diameter smaller than the membrane pore. External pollution refers to pollution caused by the sedimentation layer including sludge cake layer and gelatin layer formed by the compact combination of solid substances with the membrane through physical and chemical processes. Since external pollution is more prevalent of the two, it should be addressed preferentially.

In order to eliminate or reduce the sedimentation layer pollution (external pollution), an aeration pipe is installed inside the membrane bioreactor below the membrane module. By utilizing buoyancy of gas-water mixed flow generated during aeration, a cross-flow flushing occurs at the membrane module surface which produces water flow shearing force, and thereby, reduces the pollution of the membrane module. Meanwhile, the aeration apparatus also has the functions of supplying oxygen to the membrane bioreactor so as to keep the dissolved oxygen level (which is generally stated as D0) of pending waste water at about 2-3 mg/L to ensure or improve the efficiency of the biochemical reaction.

Micropore aeration and macropore aeration are two main aeration types that are used in membrane bioreactors. The macropore aeration can produce relatively large air bubbles and a relative stronger cross-flow flushing intensity so that a better effect to remove the sedimentation layer on the surface of the membrane module can be realized. However, the oxygen utilization rate is low (3-7%) and the energy consumption for treating waste water is high. Although micropore aeration can only produce relatively small air bubbles, it achieves oxygen utilization rate of up to 22%, and thus can significantly decrease energy consumption.

When treating highly loaded organic waste water with conventional membrane bioreactors comprising an aeration pipe installed below the membrane module, the following problems are generally encountered: (a) the increase of organic loading results in the mass propagation of micro-organisms and thus the sludge concentration increases; (b) overproduction of EPS which contributes to sedimentation of microorganisms on the surface of the membrane modules and concentration polarization; (c) the membrane pore obstruction, resulting in a decrease of the flux of the membrane module.

In order to reduce the membrane pollution due to the increase of the organic loading, the aeration rate needs to be increased, and the flow rate of the gas-water mixed flow to the surface needs to be enhanced, so as to strengthen the cross flow flushing of the gas-water mixed flow to the membrane module, to reduce the sedimentation of pollutants on the surface of the membrane, and at the same time, to keep the D0 value inside of the reaction tank at a constant level.

If macropore aeration technology is to solve the problem of membrane pollution alone, the aeration rate should be 30-60 times higher than is the case in conventional reactors. Although the micro-filtration and macro-filtration membranes have certain tensile strength, if the strength of the cross flow flushing of the mixed flow rising to the micro-filtration or macro-filtration membrane is too strong, the gas-water shearing force is correspondingly too large, and the lifespan of the membrane will be shortened largely. For example, internal pollution of the membrane yarn of the hollow-fiber membrane can result in the decrease of membrane flux and deteriorate the effluent quality.

Besides, the large aeration rate increases the energy consumption of the equipment, and in turn increases the treatment cost, so that the promotion and application of the membrane bioreactor is constrained.

On the other hand micropore aeration technology cannot solve the problem of membrane pollution alone, when treating high loaded organic waste water, the sedimentation of pollutants on the surface of the membrane module cannot be cleaned effectively; therefore, it is difficult to ensure the long-term and stable operation of the equipment.

### SUMMARY OF THE INVENTION

Therefore, it is an objective of the present invention to provide a hybrid aeration membrane bioreactor consuming less energy and being capable of stable operation wherein macropore aeration technology and micropore aeration technology are combined.

To achieve the above objective, provided is a hybrid aeration membrane bioreactor, comprising a bio-reaction tank; a plurality of membrane modules disposed within the bio-reaction tank; a macropore aeration apparatus comprising a first aeration pipe; a plurality of macropore heads having each a plurality of macropores, the macropore heads being disposed on the first aeration pipe; and a first gas pump; and a micropore aeration apparatus comprising a second aeration pipe; a plurality of micropore heads having each a plurality of micropores, the micropore heads being disposed on the second aeration pipe; and a second gas pump.

In certain embodiments of the invention, the first aeration pipe is connected to the first gas pump.

In certain embodiments of the invention, the second aeration pipe is connected to the second gas pump.

In certain embodiments of the invention, the macropore head is disposed below the membrane module.

In certain embodiments of the invention, the micropore head is disposed near the bottom of the bio-reaction tank.

In certain embodiments of the invention, the micropores are smaller in diameter than the macropores.

In certain embodiments of the invention, at least one the micropore head is disposed at a different elevation within the reactor than at least one the macropore head.

In certain embodiments of the invention, the membrane module is disposed in a center of the bio-reaction tank.

In certain embodiments of the invention, the macropore heads are spaced from one another in orderly intervals.

In certain embodiments of the invention, the macropore head is disposed immediately below the membrane module or in close proximity to the membrane module.

In certain embodiments of the invention, the micropore heads are spaced from one another in orderly intervals.

In certain embodiments of the invention, the micropore head is disposed peripherally and/or not immediately below the membrane or in close proximity to the membrane module.

In certain embodiments of the invention, the macropore heads point downwardly, i.e., the openings of the micropores are oriented towards the bottom of the tank.

In certain embodiments of the invention, the bioreactor comprises further a gas flow meter, a barometer, and/or a valve connected between the aeration pipe and the gas pump.

In certain embodiments of the invention, the micropore is 80-200 μm in diameter, and the macropore is 4-5 mm in diameter.

In accordance with the present invention, two aeration types, i.e., macropore and micropore aeration, are combined within the membrane bioreactor. Macropore aeration functions to clean the membrane surface with both gas and water so as to reduce or eliminate the sedimentation of pollutants on the membrane surface. Micropore aeration serves to keep the concentration of dissolved oxygen at a required level. Utilizing micropore aeration apparatus thus allows for the concentration of dissolved oxygen to be comparatively higher than when macropore aeration apparatus is used exclusively. On the other hand, at the same oxygen utilization rate, the rate of aeration can be lower decreasing significantly the energy consumption. Therefore, the reactor is especially suitable for treating highly loaded organic waste water.

### BRIEF DESCRIPTION OF THE DRAWINGS

FIG. 1 is a simplified structural view of a waste water treatment system comprising a hybrid aeration membrane bioreactor of the present invention;

FIG. 2 illustrates a planar view of the installation of aeration pipes of a hybrid aeration membrane bioreactor of the present invention;

FIG. 3 is a cross sectional view along the A-A line of FIG. 2; and

FIG. 4 is a cross sectional view along the B-B line of FIG. 2.

### DETAILED DESCRIPTION OF THE EMBODIMENTS

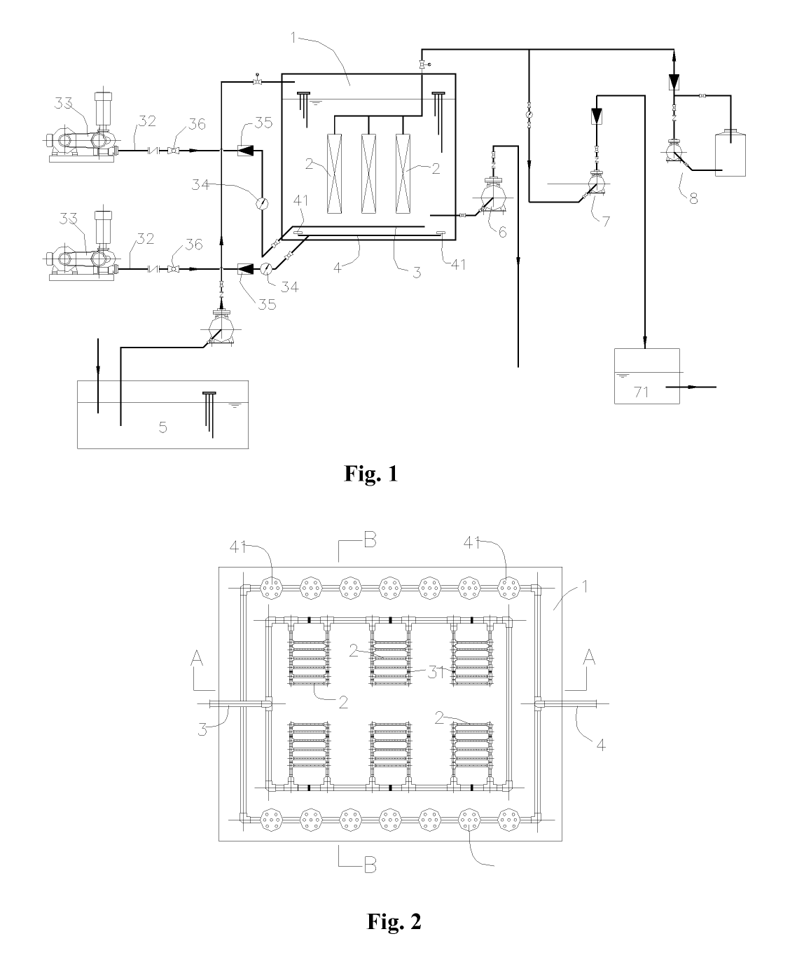

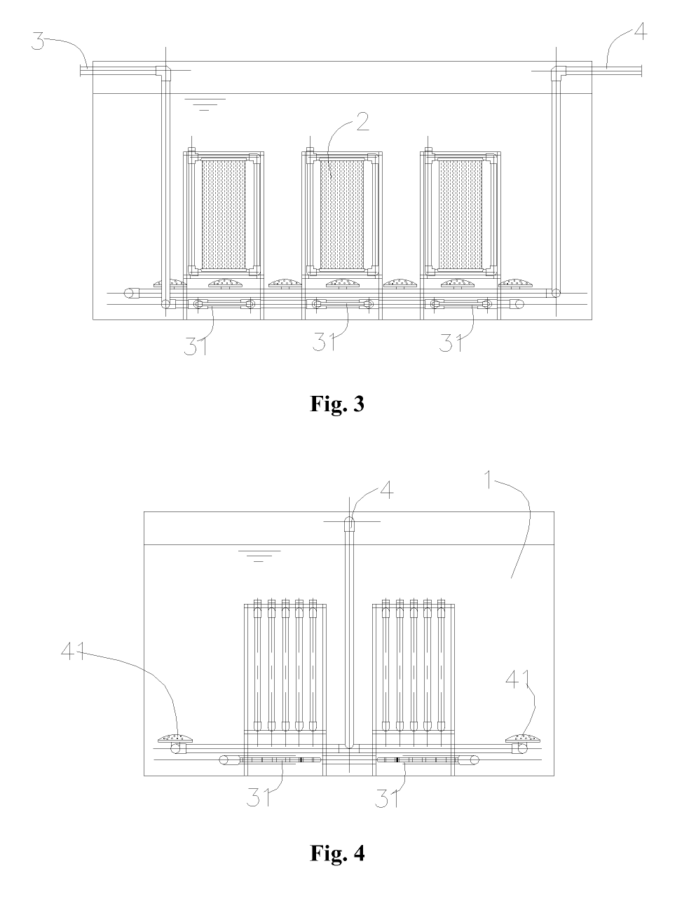

As shown in FIGS. 1-2, after removal of solid impurities, e.g., through a bar screen, highly loaded organic waste water, such as waste water from a hospital, is discharged into a retention reservoir 5, in which the waste water is homogenized and the water flow is buffered. The waste water is then lifted by a lift pump to the membrane bioreactor for treatment. Optionally, an aeration apparatus is installed in the retention reservoir to homogenize the high loaded organic waste water completely. The oxygen required in the membrane bio-reaction tank is supplied by two aeration systems, namely, the macropore aeration apparatus and the micropore aeration apparatus. Organic pollutants in the waste water are biochemically degraded and ammonia is removed in the membrane bio-reaction tank. The processed mixed liquid is filtered through and separated by an ultra-filtration membrane, and then the treated water is recycled or discharged.

In certain embodiments of the present invention, the hybrid aeration membrane bioreactor comprises a bio-reaction tank 1, an aeration device, and a plurality of membrane modules 2 installed in the bio-reaction tank and embedded below the liquid line. The membrane module 2 is an ultra-filtration or a micro-filtration membrane according to demand, such as a hollow fiber type micro-filtration membrane, an ultra-filtration membrane, or a plate type ultra-filtration membrane.

In certain embodiments, the aeration device comprises a plurality of aeration pipes installed in the bio-reaction tank 1 and a gas pump located outside the membrane bioreactor. Each aeration pipe is connected to a gas pump 33 through the pipelines 32 in a usual way. Differentiated from the existing technology, the aeration device of the present invention comprises two independent aeration pipes and a gas pump connected therewith. The first one is a macropore aeration apparatus, wherein the head 31 of each macropore aeration pipe 3 is installed below the membrane module 2 to produce relatively large bubbles so as to clean the surface of the ultra-filtration or micro-filtration membrane effectively and completely with both water and gas, and thereby, to reduce or eliminate the sedimentation of pollutants on the surface of the membrane. The other one is a micropore aeration apparatus, wherein the head 41 of each micropore aeration pipe 4 is installed at the bottom of the bio-reaction tank 1. Since the pore diameter of the head 41 of the micropore aeration pipe is smaller than that of the head 31 of the macropore aeration pipe, at the same aeration rate of gas discharge, the aeration head 41 can produce more concentrated bubbles, so that the contact area between the bubbles and the water is increased, and thereby, the oxygen is supplied at an optimum rate to the pending waste water in the tank to ensure a required level of dissolved oxygen level so as to satisfy the biochemical reaction.

Preferably, the head 41 of each micropore aeration pipe is disposed at different elevations than the head 31 of each macropore aeration pipe. Namely, the micropore aeration head 41 and the macropore aeration head 31 are not in the same elevation (shown in FIG. 2), the bubbles produced by the two different aeration pipes do not interact with each other during their production and travel to the surface so that the overall performance of the aeration apparatus is not influenced.

Generally, the membrane modules 2 are located at the center of the bio-reaction tank. A plurality of macropore aeration pipe heads 31 is disposed on the macropore aeration pipe 3 in orderly at intervals. The macropore aeration pipe heads 31 are distributed just below the membrane modules 2 or around the position just below the membrane modules 2, designed to form effectively a cross flow around the membrane modules 2 to flush the sedimentation layer on the surface of the membrane modules 2, to increase the water flow shearing force as much as possible, and to reduce or eliminate the sedimentation of pollutants on the membrane surface during the generation of macropore bubbles having large pore diameters. Optionally, a plurality of micropore aeration pipe heads is disposed orderly at intervals on the micropore aeration pipe. To avoid the arrangement of the micropore aeration pipe heads at the same elevation with the macropore aeration pipe heads 31, the micropore aeration pipe heads are distributed at the peripheral position below the bio-reaction tank, so that the supply of oxygen for the biochemical reaction of micro-organisms inside of the bio-reaction tank is ensured, and the organic substances are degraded effectively.

From the above analysis, it is clear that, at a relatively low aeration rate, the reactor of the present invention can keep the D0 value inside of the membrane bioreactor at about 2-3 mg/L, so that the required dissolved oxygen level for the biochemical reaction of micro-organisms is satisfied, and the energy consumption for treating waste water is decreased significantly.

In accordance with the membrane bioreactor of the present invention, the openings of the macropore aeration pipe heads can be oriented downwardly so as to prevent their obstruction by the sludge, and meanwhile, to ensure uniform aeration.

In further embodiments of the present invention, monitoring apparatus, such as a gas flow meter 35, a barometer 34, and a valve 36 are connected between each aeration pipe and the gas pump to adjust the aeration rate going into the membrane bioreactor according to a monitoring indication. The pump can be a normal blower. The pore diameter of the aeration head of the macropore aeration pipe is about 4-5 mm, while that of the aeration head of the micropore aeration pipe is preferably 80-200 μm.

Preferably, the ultra-filtration or micro-filtration membrane is installed in a relaxed state; in this way, the membrane yarn/face is kept vibrating when being flushed with the gas-water mixed flow, so that the efficiency to clean the sedimentation substances is enhanced.

The application of the bioreactor according to the present invention comprises the following steps: (a) lifting of waste water into the membrane bio-reaction tank by means of a lifting pump; (b) powering on the blower of the macropore aeration apparatus to flow the air into the macropore aeration pipes 3 through the aeration pipelines, a gas-water mixed uprising flow is then formed below the membrane modules 2 to generate gas-water flow shearing force so as to flush the membrane surface, to avoid the sedimentation pollution to the membrane surface, and to keep the membrane flux stable; and (c) powering on the blower of the micropore aeration apparatus to flow the air into the micropore aeration pipes 4 through the aeration pipelines, owing to the high oxygen utilization rate, the required dissolved oxygen level for the biochemical reaction of micro-organisms s ensured to degrade the organic substances efficiently.

It needs to be mentioned that, there is no special requirement on the starting sequence of above apparatuses, which can be started simultaneously or one after another, depending on the specific monitoring indication on the water quality inside of the membrane bioreactor. The sludge inside of the bio-reaction tank 1 can be removed by a sludge pump 6 periodically or occasionally. The treated water obtained through the membrane modules 2 is discharged into a clean water tank 71 by an outlet pump 7. The outlet pipeline connected with the membrane modules 2 can be further connected to a back flush unit 8 to back flush the membrane modules 2.

The hybrid aeration membrane bioreactor of the present invention utilizes a simple process, is easy to install, economical and practical, and space saving. By the combination of the macropore aeration for cleaning membrane module and the micropore aeration for supplying oxygen, the oxygen utilization rate is improved and the energy consumption is decreased. Moreover, the hybrid aeration membrane bioreactor of the present invention is capable of treating highly loaded organic waste water that cannot be treated with a conventional membrane bioreactor. Even when the waste water to be treated has a CODcr (Chemical Oxygen Demand) of between 350 mg/L and 2500 mg/L, such as is the case for a hospital wastewater, the discharge or recycling of the treated water according to a specified standard can be ensured. Therefore, the hybrid aeration membrane bioreactor of the present invention can be used widely for treating highly loaded organic waste water in various projects.

This invention is not to be limited to the specific embodiments disclosed herein and modifications for various applications and other embodiments are intended to be included within the scope of the appended claims. While this invention has been described in connection with particular examples thereof, the true scope of the invention should not be so limited since other modifications will become apparent to the skilled practitioner upon a study of the drawings, specification, and following claims.

All publications and patent applications mentioned in this specification are indicative of the level of skill of those skilled in the art to which this invention pertains. All publications and patent applications mentioned in this specification are herein incorporated by reference to the same extent as if each individual publication or patent application mentioned in this specification was specifically and individually indicated to be incorporated by reference.

What is claimed is:

1. A hybrid aeration membrane bioreactor, comprising
a bio-reaction tank;
a plurality of membrane modules disposed within said bio-reaction tank;
a macropore aeration apparatus comprising a first aeration pipe; a plurality of macropore heads having each a plurality of macropores, said macropore heads being disposed on said first aeration pipe; and a first gas pump; and
a micropore aeration apparatus comprising a second aeration pipe; a plurality of micropore heads having each a plurality of micropores, said micropore heads being disposed on said second aeration pipe; and a second gas pump;
wherein
said first aeration pipe is connected to said first gas pump;
said second aeration pipe is connected to said second gas pump;
said macropore head is disposed below said membrane module;
said micropore head is disposed near the bottom of said bio-reaction tank; and
said micropores are smaller in diameter than said macropores.

2. The bioreactor of claim 1, wherein at least one said micropore head is disposed at a different elevation within said reactor than at least one said macropore head.

3. The bioreactor of claim 1, wherein
said membrane module is disposed in a center of said bio-reaction tank;
said macropore heads are spaced from one another in orderly intervals;
said macropore head is disposed immediately below said membrane module or in close proximity to said membrane module;
said micropore heads are spaced from one another in orderly intervals; and
said micropore head is disposed peripherally and/or not immediately below said membrane or in close proximity to said membrane module.

4. The bioreactor of claim 3, wherein said macropore heads point downwardly.

5. The bioreactor of claim 4, comprising further a gas flow meter, a barometer, and/or a valve connected between said aeration pipe and said gas pump.

6. The bioreactor of claim 5, wherein said micropore is 80-200 μm in diameter, and said macropore is 4-5 mm in diameter.

7. The bioreactor of claim 2, wherein
said membrane module is disposed in a center of said bio-reaction tank;
said macropore heads are spaced from one another in orderly intervals;
said macropore head is disposed immediately below said membrane module or in close proximity to said membrane module;
said micropore heads are spaced from one another in orderly intervals; and
said micropore head is disposed peripherally and/or not immediately below said membrane or in close proximity to said membrane module.

8. The bioreactor of claim 7, wherein said macropore heads point downwardly.

9. The bioreactor of claim 8, comprising further a gas flow meter, a barometer, and/or a valve connected between said aeration pipe and said gas pump.

10. The bioreactor of claim 9, wherein said micropore is 80-200 μm in diameter, and said macropore is 4-5 mm in diameter.
